(19) United States
(12) Patent Application Publication
(10) Pub. No.: US 2024/0101951 A1
(43) Pub. Date: Mar. 28, 2024

(54) BIOLOGIC TISSUE PROCESSING DEVICE

(71) Applicant: DMS IP Assets B.B., 6411 TE Heerlen (NL)

(72) Inventors: William T. FISHER, Columbia, MD (US); Amit DEKA, Columbia, MD (US)

(21) Appl. No.: 18/266,312

(22) PCT Filed: Dec. 9, 2021
(86) PCT No.: PCT/US2021/062499

§ 371 Date: Jun. 9, 2023

Related U.S. Application Data
(60) Provisional application No. 63/123,657, filed on Dec. 10, 2020.

(51) Int. Cl. C12M 1/00; C12M 1/26; C12N 5/00

(52) U.S. Cl. CPC C12M 45/05; C12M 33/14; C12N 5/0081

(57) ABSTRACT

Disclosed herein are devices and methods for processing biologic tissues, such as adipose tissue or whole blood, via centrifugation. The devices comprise a rotatable chamber having a first outlet, a tube connected or connectable to the first outlet, a collection container in fluid communication with the tube; and a drive unit configured to rotate the rotatable chamber about the axis. The device is operable such that a sample of biologic tissue present in the rotatable chamber can be stratified into at least two constituent layers as a function of the differing specific gravities of the constituents, at least one of the constituent layers being dischargeable from the rotatable chamber via the tube and into the collection container.

### FIELD OF THE INVENTION

The present invention pertains to methods and devices for retrieving cells from biologic tissue, such as retrieving adipose-derived stem cells from adipose tissue.

### BACKGROUND

Adipose tissue, also called fat or fat tissue, is composed mostly of adipocytes. Other cell types are present, collectively termed stromal vascular fraction (SVF) cells. The SVF includes multiple cell types, including Adipose-derived stem cells (ADSCs).

ADSCs are stem cells obtained from adipose tissue. ADSCs have also been described as adipose-derived stem/stromal cells, adipose-derived adult stem cells, adipose-derived adult stromal cells, adipose-derived stromal cells, adipose stromal cells, adipose mesenchymal stem cells, lipoblast, pericyte, preadipocyte, adipose stem cells, and processed lipoaspirate cells. ADSCs are known to be useful in various medical procedures to assist in the healing of an affected area of a patient, for example by providing enhanced cellular regeneration of a treatment site.

ADSCs can be obtained from adipose tissue in a multi-step process: fat tissue is extracted from the patient, the ADSCs are liberated from the fat tissue, and the ADSCs are recovered by separating them from other components of the fat tissue. Fat tissue is extracted from a patient using known techniques, such as surgery or liposuction. Existing techniques to liberate ADSCs utilize enzymes to break down the adipose tissue. The cells are then separated, usually by centrifuge, sedimentation or filtration techniques. The separated cells are usually then washed to remove the enzyme (residuals) used to treat the fat sample. Disadvantageously, the process to prepare a useful sample of ADSCs using presently available techniques takes several hours (and in some cases up to 14 days), making the ad-hoc use of such a procedure difficult or impossible. In addition, the techniques require multiple processing steps, thereby increasing the potential for contamination.

Mechanical separation and extraction of components of adipose tissues is a possible alternative to existing techniques. A centrifuge device for obtaining ADSCs is disclosed in WO2015/117007. In certain disclosed embodiments, the act of centrifugation is used to both liberate the ADSCs from a fat tissue sample and separate the ADSCs from other material once liberated.

Further examples of potentially relevant devices are disclosed in WO2012/006587, WO2012/067658, WO2013/106655, WO2013/123216, US20140021147, WO2014/011213, WO2014/0164815, WO2014/039697, WO2014/110448, WO2014/154990, US2015093362, WO2015/035221, and WO2018/044791.

### SUMMARY

The inventors have discovered that the forces that may be required to liberate and separate ADSCs from other material via centrifugation may destroy the ADSCs in the collection process. There is thus a need for improved devices and processes for liberating, separating, and collecting ADSCs from fat tissue. The disclosed devices and methods may also have utility in separation and extraction of components of other biologic tissues, such as blood.

In an embodiment, a device for processing a biologic tissue comprises:
a. a rotatable chamber arranged to rotate around an axis, the rotatable chamber comprising a first end, a second end, a sidewall extending between the first end and the second end and comprising an inner surface that decreases in diameter over at least a portion of the distance between the first end and the second end, and a first outlet, the first outlet beginning in the interior of the rotatable chamber at a first radial distance from the axis;
b. a tube connected or connectable to the first outlet, the tube extending in a direction that is at least radially away from the axis over at least part of the tube's length;
c. a collection container in fluid communication with the tube; and
d. a drive unit configured to rotate the rotatable chamber about the axis;
wherein the device is operable such that a centrifugal field is produced via rotation of the rotatable chamber about the axis, thereby causing a sample of biologic tissue present in the rotatable chamber to stratify into at least two constituent layers as a function of the differing specific gravities of the constituents, at least one of the constituent layers being dischargeable from the rotatable chamber via the tube and into the collection container, wherein an interior wall of the collection container and the trajectory of the exit of the tube are configured to reduce the velocity of the constituent layer at the time the constituent layer contacts the interior wall of the collection container relative to the velocity of the constituent layer at the time that the constituent layer exits the tube.

The disclosed inventions may be beneficial over the prior art in that they may extract cells from a sample at a greater percentage of viability, be more sterile, more self-contained, faster, more compact, easier to use, better at removing contaminants, better at removing oils or other liquids, or otherwise more efficacious than the prior art devices.

### BRIEF DESCRIPTION OF THE DRAWINGS

FIG. 1 is an exploded view of a processing unit of a device according to an embodiment of the invention.

FIG. 2 is a partial cross-section view of the processing unit depicted in FIG. 1.

FIG. 3 is an overhead view of the processing unit depicted in FIG. 1.

### DESCRIPTION

The device comprises a rotatable chamber. The rotatable chamber is arranged to rotate around a central axis. In an embodiment, the axis is the longitudinal axis of the rotatable chamber. The rotatable chamber comprises a first end and a second end, with a sidewall comprising an inner surface that decreases in diameter, or decreases in distance from the axis, over at least a portion of the distance between the first end and the second end. Additional inner surfaces may be present, for example, an inner surface that is parallel to the axis extending a portion of the distance from the first end to the second end. In an embodiment, the first end has a diameter smaller than the second end.

An inner surface urges the liquid to move toward an outlet of the rotatable chamber. In an embodiment, the rotatable chamber has a first end and a second end, with a sidewall extending therebetween, wherein said first end has a diameter smaller than a diameter of said second end. In an embodiment, the second end is the base of the rotatable chamber. In an embodiment, the second end is the base. In an embodiment, the largest radial distance from the axis to the sidewall in the interior of the rotatable chamber is proximate the second end, such as at the intersection of the second end and the sidewall. The sidewall and/or rotatable chamber may be fully or partially transparent to allow for visual inspection of the sample and centrifugation.

In an embodiment, the sidewall is uniformly angled from the first end to the second end. In an embodiment, the sidewall is angled at multiple different angles along its length. For example, in an embodiment the sidewall is angled in a first direction and a second direction, wherein the first direction is away from the first end, towards the second end, and away from the axis, and wherein the second direction is away from the first end, towards the second end, and towards the axis. In an embodiment, at least a portion of the sidewall is curved.

In an embodiment, the device comprises an outer enclosure surrounding the rotatable chamber. The rotatable chamber may be contained within an outer enclosure to ensure that the operator cannot inadvertently touch the rotatable chamber. The outer enclosure may be arranged to receive a biologic mixture from the rotatable chamber and may be arranged coaxially upon the central axis of the rotatable chamber. In an embodiment, the outer enclosure encloses at least the rotatable chamber and the collection container.

The rotatable chamber may comprise an inlet to allow a sample, e.g. lipoaspirate, to be placed into the rotatable chamber. In an embodiment, the inlet is configured to receive the tip of a syringe or catheter. In an embodiment, the inlet comprises a cap that can be opened to allow input of the sample and closed to seal the rotatable chamber or outer enclosure. In an embodiment, the rotatable chamber incorporates one or more vents to allow for fluid displacement. One or more vents may provide for air to enter the chamber from which a fluid is displaced, or for air to leave a chamber from which fluid enters.

The rotatable chamber comprises a first outlet. In an embodiment, the first outlet is located proximate the base of the rotatable chamber, such as in the sidewall near the base of the rotatable chamber, in the base of the rotatable chamber proximate the sidewall, or at the intersection of the base and the sidewall. In an embodiment, the first outlet begins at the greatest diameter of the interior volume of the rotatable chamber. In an embodiment, the first outlet extends through the sidewall from the interior of the rotatable chamber to the exterior of the rotatable chamber.

In an embodiment, the rotatable chamber comprises a channel as a portion of the first outlet. In an embodiment, the first outlet comprises a channel and a first port in the channel, wherein the first port is in fluid communication with the channel and comprises a section of the first outlet that extends through the rotatable chamber to an exterior of the rotatable chamber or to the tube. In an embodiment, the first port is a thru hole in the wall of the rotatable chamber. The channel is a passage within the rotatable chamber prior to any portion of the first outlet that extends through the sidewall or base of the rotatable chamber. In an embodiment, the channel is a circumferential channel, extending along the circumference of the rotatable chamber proximate the sidewall. The channel may be defined at least partially by a wedge, such as an annular wedge, located within the interior of the chamber. In such case the channel would be defined by the outer surface of the annular wedge and the sidewall.

The channel may be of uniform width or may comprise a restriction. The restriction may be uniform along the length of the channel, causing the width of the restriction to decrease uniformly from a maximum width to a minimum width. The restriction may also be present over a certain length (along the circumference) and/or height of the channel. The restriction may be configured to restrict flow of fluid attempting to flow into the channel and/or restrict the flow of fluid in the channel. The restriction may be present over a certain distance over a circumferential channel, such as from 20 to 180 degrees of the circumference of the circumferential channel.

The device comprises a tube connected or connectable to the first outlet. The tube may be connected to the first outlet by, for example, opening a valve or axially translating a valve cap that comprises a thru hole that aligns the first outlet and the tube. The tube is generally present at a greater radial distance than the first outlet. When the tube is connected to the first outlet and an associated valve is opened, fluid is able to flow from the interior of the rotatable chamber into the collection container.

The tube may be part of the rotatable chamber itself or part of a valve cap that may be translated to align the inlet of the tube with the exit of the first outlet, thereby opening or closing the valve. In such cases, the tube would rotate along with the chamber. In other embodiments, the tube may remain stationary.

Due to the configuration of the tube and wall of the collection container, the constituent layer's velocity when the constituent layer contacts a wall of the collection container is substantially less than the velocity of the constituent layer as it exits the tube. In an embodiment, this is achieved because the tube discharges into the collection container at a direction that is substantially parallel to the direction of the perimeter of the interior wall of the collection container. In an embodiment, the tip of the tube, as it rotates, traces a path as it rotates around the axis that it substantially parallel to an interior wall of the collection container. In an embodiment, the direction of discharge out of the tube is substantially tangential to a circle centered about the axis in the plane of the circle or is parallel to a tangent line of an interior wall of the collection container along the same radial direction from the axis.

In an embodiment, the distance from the end of the tube to the nearest wall of the collection container along the direction of discharge out of the tube is at least 3, 4, 5, 10, 15, 20, or 25 times the internal diameter of the end of the tube at a given position of the tube about the axis. In an embodiment, the distance from the end of the tube to the nearest wall of the collection container along the direction of discharge out of the tube is at most 200, 150, 100, 75, 50, 25, 20, 15, or 10 times the internal diameter of the end of the tube at a given position of the tube about the axis. In an embodiment, the distance from the end of the tube to the nearest wall of the collection container along the direction of discharge out of the tube is at least 3, 4, 5, 10, 15, 20, or 25 times the internal diameter of the end of the tube at any given position of the tube about the axis. In an embodiment, the distance from the end of the tube to the nearest wall of the collection container along the direction of discharge out of the tube is at most 200, 150, 100, 75, 50, 25, 20, 15, or 10 times the internal diameter of the end of the tube at any given position of the tube about the axis. In an embodiment, the distance from the end of the tube to the nearest wall of the collection container along the direction of discharge out of the tube is at least 15 mm, 20 mm, 25 mm, 30 mm, 35 mm, 40 mm, 45 mm, 50 mm, 55 mm, 60 mm, 65 mm, 70 mm, or 75 mm.

In an embodiment, the velocity of the constituent layer at the time it contacts a wall of the collection container is 90% or less, 80% or less, 70% or less, 60% or less, 50% or less, 40% or less, or 30% or less of the velocity of the constituent layer at the point it is discharged from the tube. In an embodiment, the velocity of the constituent layer at the time it contacts a wall of the collection container is from 20%, 30%, 40%, 50%, 60%, 70%, or 80% to 90% of the velocity of the constituent layer at the point it is discharged from the tube.

The dimension and direction of the tube itself may take various forms. For instance, the internal cross-sectional area of the tube may vary, such that it is greater at its exit than at its entrance, increases over its length, increases at a uniform rate over its length.

The tube may comprise various portions over its length. Such portions may take varying trajectories. For example, the tube may extend in a direction that is at least axially along the axis, or in a direction that is at least radially away from the axis, or both. In an embodiment, the tube extends in a direction axially along the axis along the entire length of the tube.

In an embodiment, the tube comprises multiple portions. The multiple portions may be separated by a bend wherein the trajectory of the tube changes. In an embodiment, a first portion and a second portion of the tube are separated by a bend of approximately 90 degrees. In an embodiment, a first portion of the tube extends from the entrance of the tube to a second portion, or bend separating the first portion and the second portion. In an embodiment, the second portion extends from the first portion, or bend separating the first portion and the second portion, to the exit of the tube. In an embodiment, a first portion extends in a direction that is at least radially away from the axis. In an embodiment, a first portion extends in a direction that is both radially away from the axis and axially along the axis. In an embodiment, a second portion extends in a direction that is at least axially along the axis. In an embodiment, the second portion extends in a direction that is both radially away from the axis and axially along the axis. In an embodiment, the second portion extends in a circular direction, in other words in the trajectory of a circle, whether having its center at the axis or elsewhere. In an embodiment, the second portion extends in a direction circularly around the axis, in other words in the trajectory of a circle centered at the axis. In an embodiment, the second portion extends in a circular direction, axially along the axis, and radially away from the axis. In an embodiment, the first portion extends in a direction radially away from the axis and axially along the axis and the second portion extends in a direction circularly around the axis and axially along the axis. In an embodiment, the first portion extends in a direction radially away from the axis and axially along the axis and the second portion extends in a circular direction and axially along the axis.

In an embodiment, the first outlet is in fluid communication with a first valve that determines whether liquid may flow into the tube from the first outlet. In an embodiment, the valve is manually operable by the user. In an embodiment, the valve operates automatically as a result of the operation of the device.

In an embodiment, the valve is opened or closed by axially translating a valve cap positioned between the first outlet and the tube. The valve cap comprises a thru hole that, when positioned appropriately, puts the first outlet and the tube in fluid communication.

In an embodiment, the valve comprises a dynamic seal. A dynamic seal is a seal that is normally in the closed position, thereby restricting flow through the channel, and opens in response to the force exerted on the dynamic seal. The force may be exerted on the dynamic seal by the sample or may be exerted on the dynamic seal by the rotation of the device. In an embodiment, the dynamic seal comprises an o-ring positioned in the channel.

The collection container collects material that is expelled from the rotatable chamber, under force exerted on the material by the centrifugal field. In an embodiment, the device comprises a collection container. In an embodiment, a collection container is contained within the device or within the outer enclosure. In an embodiment, the collection container is separately removable from the device. In an embodiment, the collection container comprises an annular shape such that it fits around the rotatable chamber, and other optional components such as an endcap, valve cap or an enclosure. The collection container may have an opening in one of its walls through which the tube may be positioned. In the case that the tube rotates while the collection container remains stationary, the opening may be a circular groove. In an embodiment, the tube protrudes from a wall of the collection container, such as being formed as part of an interior wall of the collection container.

In an embodiment, the rotatable chamber further comprises a second outlet at a different radial distance from the axis than the first outlet. The second outlet may be the opening of a second channel. The second channel may comprise a second valve. Further outlets, channels, and valves may also be present.

In an embodiment, the inner chamber comprises a trap. A trap is region at the bottom of the rotatable chamber that traps certain constituents of the sample during rotation of the rotatable chamber such that the trapped constituents are inhibited from remixing with other constituents of the sample after rotation is stopped. In an embodiment, the rotatable chamber comprises an oil trap for trapping oil near the base of the rotatable chamber.

In an embodiment, a filter is positioned at a distance above the base of the rotatable chamber, thereby forming a trap. The filter may be useful to keep the constituent of biologic tissue separate from any oil or lipid fraction that may have been separated by the centrifugation. For example, in an embodiment wherein a retaining screen is present, a sample of adipose tissue may separate into the following constituents: internatant fluid, adipose tissue, and oil and lipid fraction. Generally, the oil and lipid fraction will be present nearest the axis of rotation of the rotatable chamber. However, due to the presence of the retaining screen, a first portion of the oil and lipid fraction will pass through the retaining screen and be present adjacent the internatant fluid and a second portion of the oil and lipid fraction will not pass through the retaining screen and be present on the interior surface of the adipose tissue, nearer the axis of rotation of the rotatable chamber. The first portion of the oil and lipid fraction may be ejected through the first outlet along with the internatant fluid. When the rotation is stopped the second portion of oil and lipid fraction may undesirably remix with the adipose tissue if the filter is not present. The filter serves to keep the second portion of the oil and lipid fraction separate from the adipose tissue because the oil and lipid fraction will pass through the filter due to gravity whereas the adipose tissue will not pass through the filter.

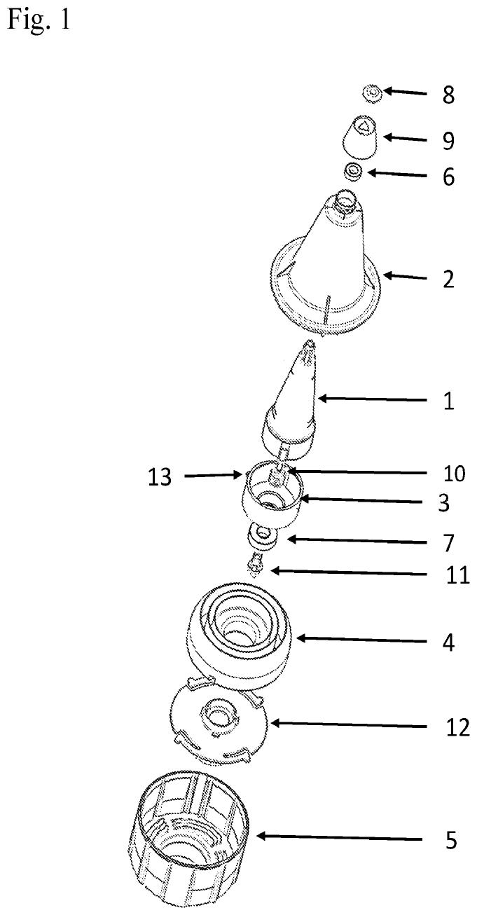

In an embodiment, the filter is shaped like a disc. Such an embodiment is shown in FIG. 1. In an embodiment, the filter is made of metal, such as stainless steel. In an embodiment, the filter is made of a plastic, such as nylon. In an embodiment, the filter has an average opening size is of at least 0.025 mm or 0.05 mm. In an embodiment, the filter to has an average opening size of at most 0.25 mm, 0.2 mm, 0.15 mm, or 0.13 mm.

The rotatable chamber may comprise a fiber collector as disclosed in WO2020/077097. The fiber collector is configured to collect fibers from the sample, such as collagen fibers in lipoaspirate. A wiper for disrupting fibers hanging off of the fiber collector may also be present.

The rotatable chamber may further comprise one or more screens, such as a morselizing screen or a retaining screen, in its interior. A morselizing screen is a screen configured to break apart a sample, such as to liberate a certain type of cells. Generally, a morselizing screen allows for portions of the sample to pass through the morselizing screen, whereas a retaining screen allows for portions of the sample to be retained on the surface of the retaining screen nearest the axis.

In an embodiment, the rotatable chamber further comprises a morselizing screen. The morselizing screen is configured to morselize tissue into smaller fragments. In an embodiment, the act of morselizing comprises breaking a plurality of adipocytes present in adipose tissue. A morselizing screen may be useful for, for example, extracting multipotent stem cells, such as ADSCs, from adipose tissue.

In an embodiment the morselizing screen is cylindrical. In an embodiment, a surface of the morselizing screen is substantially parallel to an inner surface of the sidewall. In an embodiment, the morselizing screen is frustoconical. In an embodiment, the morselizing screen rotates along with the rotatable chamber. In an embodiment, the morselizing screen is stationary relative to the rotatable chamber. In an embodiment, the morselizing screen rotates at a revolution frequency that is less that the revolution frequency of the rotatable chamber. In an embodiment, the morselizing screen projects away from the base. In an embodiment, the morselizing screen extends concentrically around the axis of the rotatable chamber. In an embodiment, the retaining screen is mesh-like. The morselizing screen may be a metal or polymer wire material, or a perforated sheet having openings of sufficient size to at least partially morselize the sample.

Although the purpose of the morselizing screen is to morselize the tissue, the tissue may not immediately morselize when it comes into contact with the morselizing screen depending, for instance, on the type of tissue. The tissue may instead be somewhat retained by the morselizing screen. It may be advantageous to urge the tissue through the morselizing screen in order to morselize the tissue. In an embodiment, the tissue is urged away from the axis of rotation of the rotatable chamber and toward the sidewall. In an embodiment, the device further comprises a roller arranged to urge tissue through the morselizing screen. In an embodiment, the roller comprises a roller axle and a cylinder rotatable about the roller axle. In an embodiment, the roller axle is formed of stiff wire that extends through the center bore of the cylinder and the wire is mounted so that it may be secured in a stationary position within the inner chamber.

In an embodiment, the roller is present proximate or in contact with the morselizing screen. As either the roller axle or morselizing screen rotate about the axis of rotation of the rotatable chamber along with the rotatable chamber, the roller may come into contact with tissue built up on the interior surface of the morselizing screen, thereby urging the tissue through the morselizing screen. In an embodiment, the morselizing screen is rotatable while the roller axle remains stationary. In an embodiment, the roller is present near the inner surface of the morselizing screen but does not contact the morselizing screen. In an embodiment, the roller rolls against the inner surface of the annular screen.

In an embodiment, the rotatable chamber further comprises a retaining screen. The retaining screen is configured to substantially retain a certain type of tissue on the interior of the retaining screen while allowing liquids to pass through. A retaining screen may serve to keep obstructions in the form of biologic tissue from reaching the outlet. In an embodiment, the retaining screen is configured to retain fat tissue on its inner surface. In an embodiment the retaining screen is cylindrical. In an embodiment, a surface of the retaining screen is substantially parallel to an inner surface of the sidewall. In an embodiment, the retaining screen is frustoconical. In an embodiment, the retaining screen rotates along with the rotatable chamber. In an embodiment, the retaining screen is stationary relative to the rotatable chamber. In an embodiment, the retaining screen rotates at a revolution frequency that is less that the revolution frequency of the rotatable chamber. In an embodiment, the retaining screen projects away from the base. In an embodiment, the retaining screen extends concentrically around the axis of the rotatable chamber. In an embodiment, the retaining screen is mesh-like. The retaining screen may be a metal or polymer wire material, or a perforated sheet having openings of sufficient size to allow for the passage of fluid while inhibiting desirable material to pass through.

In an embodiment, the device comprises a drive unit, which serves as a rotation source for the rotatable chamber. Upon reaching a sufficient rotation speed, a biologic tissue sample stratifies into at least two constituents based on the density of the constituents. The drive unit preferably couples to the rotatable chamber and/or outer enclosure. The drive unit comprises means to rotate the rotatable chamber. The drive unit preferably comprises an electric motor configured to rotate the rotatable chamber, but may also contains a hand crank or any other means to rotate the rotatable chamber that may be known to a person skilled in the art. In an embodiment, the drive unit comprises a hand crank and a spring and is configured so that the drive unit can be would up and the spring released to rotate the rotatable chamber. In an embodiment, the drive unit is separable from the rotatable chamber, such that the drive unit may be reused without substantial cleaning or sterilization. In an embodiment, the rotatable chamber, and optionally the outer enclosure, are single-use or may be cleaned and sterilized, such that they may be reused.

In an embodiment, the device comprises two units: a processing unit and a drive unit. The drive unit comprises the base of the unit and rotation source. The processing unit comprises the upper portion of the device and comprises the rotatable chamber, tube, and collection container. The processing unit generally single use, though it may be sterilized and reused, and the drive unit is reusable without sterilization.

In an embodiment, the drive unit is configured to rotate the rotatable chamber at multiple speeds. In an embodiment, the drive unit is configured to rotate the rotatable chamber such that the g-force acting on a sample within the rotatable chamber is 1000 g or less, 8000 g or less, or 5000 g or less. In an embodiment, the drive unit is configured to oscillate the rotatable chamber, such as a washing machine. In an embodiment, the drive unit is configured to oscillate in a sinusoidal wave pattern. In an embodiment, the drive unit is configured to oscillate in a square wave pattern. The drive unit may comprise manually operated controls, such as buttons, to allow an operator to direct the drive unit to rotate the rotatable chamber in a certain way, such as at a certain direction or certain speed.

FIG. 1 depicts an exploded view of a separation unit according to an embodiment of the invention. The depicted separation unit principally comprises rotatable chamber 1, chamber enclosure 2, valve cap 3, collection container 4, and collar 5. The separation unit also comprises various elements to interface the components of the device, including bearings 6, 7, inlet seal 8, inlet cap 9, spring 10, drive gear 11, and chamber base 12. Chamber enclosure 2 couples to collar 5 to form an outer enclosure around the rotatable chamber. Removing the chamber enclosure 2 allows access to the collection container 4, which sits on chamber base 12. However, it may be preferable that the outer enclosure and collection container comprise a port that allows for access to the contents of the collection container 4 without removal of chamber enclosure 2. As depicted, valve cap 3 comprises tube 13.

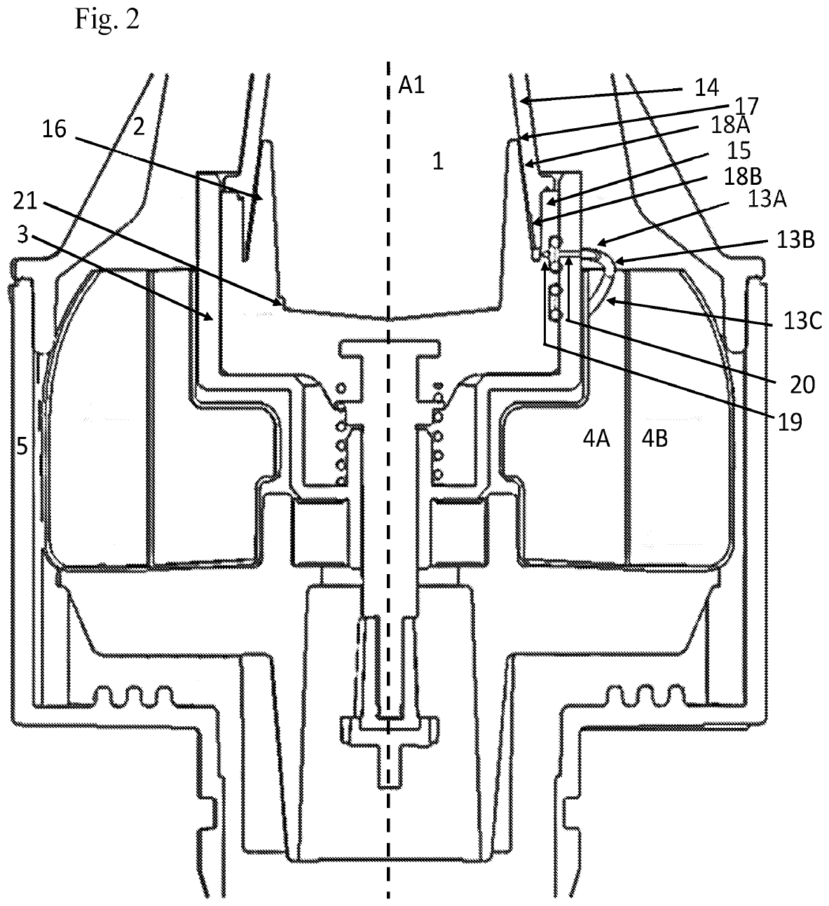

FIG. 2 depicts a cross-section of the assembled separation unit of FIG. 1. Chamber enclosure 2 couples with collar 5 to form the outer enclosure. Rotatable chamber 1 comprises sidewall 14, endcap 15, and wedge 16. The first outlet begins at position 17. The first outlet is defined by channel 18 and first port 19. The channel is circumferential and defined by the outer surface of wedge 16 and the inner surface of sidewall 14. The channel 18 is restricted over portion 18a and unrestricted over portion 18b, resulting in a reduction in width of the channel in the restricted portion 18a. First port 19 extends through the endcap 15 to the valve cap 3. In operation, valve cap 3 spins along with rotatable chamber 1. When first port 19 is aligned with first thru hole 20 in valve cap 3, tube 13 is in fluid communication with the first outlet and fluid may be dispensed from rotatable chamber 1 into an inner portion of the collection container 4A.

As depicted, tube 13 comprises a first portion 13a, a bend 13b, and a second portion 13c. First portion 13a extends radially from axis A-1. Second portion 13c extends radially and circularly around axis A-1. Tube 13 discharges into an inner section of the collection container 4A.

The rotatable chamber may further comprise a second port, such as one beginning at location 21. The second port may extend through end cap 15 and wedge 16. A second thru hole in the valve cap 3 may be aligned with a second port to form a second outlet that may discharge a second fraction from the rotatable chamber. As depicted, the collection container is segmented into an inner section 4A and outer section 4B such that a first fraction is discharged through the first outlet into a first section of the collection container 4A and a second fraction is discharged through the second outlet into a second section of the collection container 4B.

The rotatable chamber depicted does not comprise a morselizing screen or retaining screen. If present both screens would be present between axis A-1 and the beginning of the first outlet, with the morselizing screen having a lesser radial distance from the axis than the retaining screen.

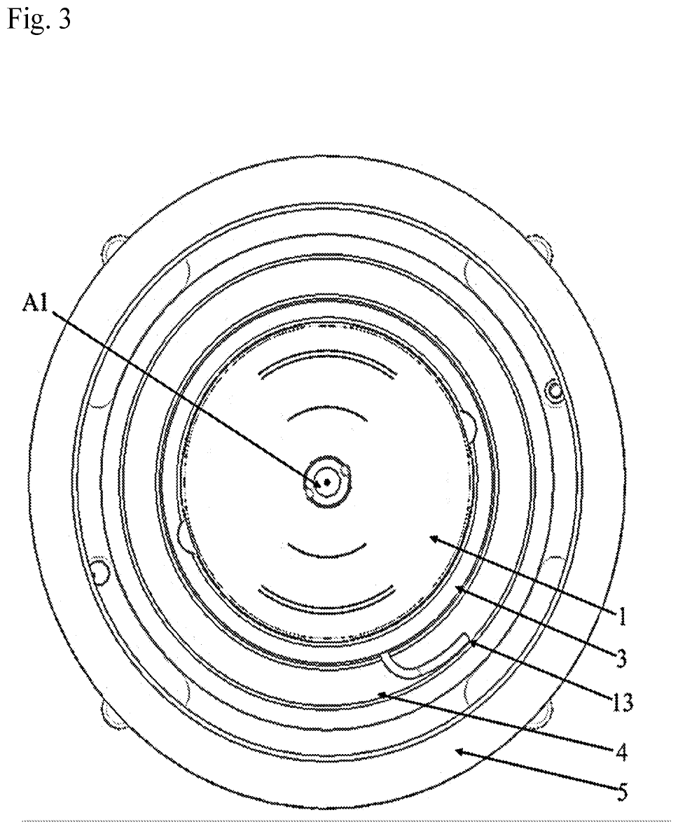

Turning now to FIG. 3, the processing unit of a device according to an embodiment of the invention is pictured from an overhead view. Rotatable chamber 1 is in contact with valve cap 3, collection container 4, and collar 5. Tube 13 extends circularly about axis A1 and axially into the page as pictured. The direction of discharge from tube 13 is substantially tangential to a circle centered about axis A1 and parallel to the direction of an interior wall of the collection container.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

### ADDITIONAL DESCRIPTION OF CERTAIN EXEMPLARY EMBODIMENTS

1. A device for processing biologic tissue, the device comprising:
   a. a rotatable chamber arranged to rotate around an axis, the rotatable chamber comprising a first end, a second end, a sidewall extending between the first end and the second end and comprising an inner surface that decreases in diameter over at least a portion of the distance between the first end and the second end, and a first outlet, the first outlet beginning in the interior of the rotatable chamber at a first radial distance from the axis;
   b. a tube connected or connectable to the first outlet, the tube extending in a direction that is at least radially away from the axis over at least part of the tube's length;
   c. a collection container in fluid communication with the tube; and
   d. a drive unit configured to rotate the rotatable chamber about the axis;
   wherein the device is operable such that a centrifugal field is produced via rotation of the rotatable chamber about the axis, thereby causing a sample of biologic tissue present in the rotatable chamber to stratify into at least two constituent layers as a function of the differing specific gravities of the constituents, at least one of the constituent layers being dischargeable from the rotatable chamber via the tube and into the collection container, wherein an interior wall of the collection container and the trajectory of the exit of the tube are configured to reduce the velocity of the constituent layer at the time the constituent layer contacts the interior wall of the collection container relative to the velocity of the constituent layer at the time that the constituent layer exits the tube.
2. A device for processing biologic tissue, the device comprising:
   a. a rotatable chamber arranged to rotate around an axis, the rotatable chamber comprising a first end, a second end, a sidewall extending between the first end and the second end and comprising an inner surface that decreases in distance from the axis over at least a portion of its length between the first end and the second end, and a first outlet, the first outlet beginning in the interior of the rotatable chamber at a first radial distance from the axis;
   b. a tube connected or connectable to the first outlet, the tube extending in a direction that is at least radially away from the axis over at least part of the tube's length;
   c. a collection container in fluid communication with the tube; and
   d. a drive unit configured to rotate the rotatable chamber about the axis;
   wherein the device is operable such that a centrifugal field is produced via rotation of the rotatable chamber about the axis, thereby causing a sample of biologic tissue present in the rotatable chamber to stratify into at least two constituent layers as a function of the differing specific gravities of the constituents, at least one of the constituent layers being dischargeable from the rotatable chamber via the tube and into the collection container,
   wherein an interior wall of the collection container and the trajectory of the exit of the tube are configured to reduce the velocity of the constituent layer at the time the constituent layer contacts the interior wall of the collection container relative to the velocity of the constituent layer at the time that the constituent layer exits the tube.
3. The device according to any one of the preceding exemplary embodiments, wherein the biologic tissue is blood.
4. The device according to any one of the preceding exemplary embodiments, wherein the biologic tissue is adipose tissue.
5. The device according to any one of the preceding exemplary embodiments, wherein the device is operable to separate and retain adipose-derived stem cells from adipose tissue.
6. The device according to any one of the preceding exemplary embodiments, wherein the device is operable to liberate, separate, and retain adipose-derived stem cells from adipose tissue.
7. The device according to any one of the preceding exemplary embodiments, wherein “in fluid communication” means that fluid makes the connection between the parts specified, that the parts are connected by fluid, or that fluid may flow from one part specified to another part specified.
8. The device according to any one of the preceding exemplary embodiments, wherein the rotatable chamber is tubular.
9. The device according to any one of the preceding exemplary embodiments, wherein the second end is the base of the rotatable chamber.
10. The device according to any one of the preceding exemplary embodiments, wherein said first end has a diameter smaller than a diameter of said second end.
11. The device according to any one of the preceding exemplary embodiments, wherein the rotatable chamber is rotatable about a longitudinal axis of the rotatable chamber.
12. The device according to any one of the preceding exemplary embodiments, wherein the sidewall is angled in a first direction and a second direction, wherein the first direction is away from the first end, towards the second end, and away from the axis, and wherein the second direction is away from the first end, towards the second end, and towards the axis.
13. The device according to the previous exemplary embodiment, wherein the largest diameter of the rotatable chamber is present at a location on the sidewall between the first direction and the second direction.
14. The device according to any one of the preceding exemplary embodiments, wherein the largest distance from the axis in the rotatable chamber is proximate the second end.
15. The device according to any one of the preceding exemplary embodiments, wherein the largest distance from the axis in the rotatable chamber is at the intersection of the sidewall and the second end.
16. The device according to any one of the preceding exemplary embodiments, wherein the rotatable chamber is at least partially transparent.
17. The device according to any one of the preceding exemplary embodiments, further comprising an outer enclosure enclosing the rotatable chamber.
18. The device according to any one of the preceding exemplary embodiments, wherein the outer enclosure is arranged to receive a biologic mixture from the rotatable chamber.
19. The device according to any one of the preceding exemplary embodiments, wherein the outer enclosure is arranged coaxially upon the axis of rotation of the rotatable chamber.
20. The device according to any one of the preceding exemplary embodiments, wherein the rotatable chamber comprises an inlet to allow a sample to be placed into the rotatable chamber.
21. The device according to any one of the preceding exemplary embodiments, wherein the inlet is configured to receive the tip of a syringe.
22. The device according to any one of the preceding exemplary embodiments, wherein the inlet is configured to receive a catheter.
23. The device according to any one of the preceding exemplary embodiments, wherein the inlet comprises a cap that can be opened to allow input of the sample and closed to seal the rotatable chamber and/or outer enclosure.
24. The device according to any one of the preceding exemplary embodiments, wherein the first outlet begins in the sidewall proximate the base of the rotatable chamber, begins in the base of the rotatable chamber proximate the sidewall, or begins at the intersection of the base of the chamber and the sidewall.
25. The device according to any one of the preceding exemplary embodiments, wherein the first outlet begins at the greatest diameter of the interior volume of the rotatable chamber.
26. The device according to any one of the preceding exemplary embodiments, wherein the first radial distance is the maximum distance from the axis within the rotatable chamber.
27. The device according to any one of the preceding exemplary embodiments, wherein the first outlet extends through the sidewall from the interior of the rotatable chamber to the exterior of the rotatable chamber.
28. The device according to any one of the preceding exemplary embodiments, wherein the first outlet comprises a channel extending from the interior of the rotatable chamber.
29. The device according to any one of the preceding exemplary embodiments, wherein the first outlet comprises a circumferential channel extending from the interior of the rotatable chamber.
30. The device according to any one of the preceding exemplary embodiments, wherein the first outlet comprises a channel extending from the interior of the rotatable chamber, wherein the channel is defined by a wall of a wedge located within the interior of the chamber and the sidewall.
31. The device according to any one of the preceding exemplary embodiments, wherein the rotatable chamber further comprises an annular wedge located within said rotatable chamber and projecting from the second end toward the first end, said annular wedge having a wall located adjacent said sidewall of said barrel to define therebetween a channel.
32. The device according to any one of the preceding exemplary embodiments, wherein the channel comprises a restriction configured to at least partially restrict fluid flow into the channel from the interior of the rotatable chamber over some portion of the channel.
33. The device according to any one of the preceding exemplary embodiments, wherein the restriction is present at the entrance of the channel from the interior of the rotatable chamber.
34. The device according to any one of the preceding exemplary embodiments, wherein the restriction is configured to restrict fluid flow into the circumferential channel in a direction from a surface of the wedge that is nearest the first end.
35. The device according to any one of the preceding exemplary embodiments, wherein the restriction is configured to restrict fluid flow into the circumferential channel over from about 20 to 180 degrees of the circumference of the circumferential channel.
36. The device according to any one of the preceding exemplary embodiments, wherein the restriction is configured to provide a variable reduction in the width of the circumferential channel in a direction radially from the axis.
37. The device according to any one of the preceding exemplary embodiments, wherein the restriction is configured to provide a variable reduction in the height of the circumferential channel in a direction at least partially axially along the axis.
38. The device according to any one of the preceding exemplary embodiments, wherein the first outlet comprises a channel and a first port in the channel, wherein the first port is in fluid communication with the channel and comprises a section of the first outlet that extends through the rotatable chamber to an exterior of the rotatable chamber or to the tube.
39. The device according to any one of the preceding exemplary embodiments, wherein the greatest restriction in the channel is at the portion of the channel nearest the first port.
40. The device according to any one of the preceding exemplary embodiments, wherein the fluid flow in a direction circumferentially around the circumferential channel is unrestricted over at least a portion of the height of the circumferential channel.
41. The device according to any one of the preceding exemplary embodiments, wherein the least restriction in the channel is at the portion of the channel nearest the first port.
42. The device according to any one of the preceding exemplary embodiments, wherein the tube is connected or connectable to the first outlet and at a distance greater than the first radial distance.
43. The device according to any one of the preceding exemplary embodiments, wherein the device is configured such that the tube rotates along with the rotatable chamber.
44. The device according to any one of the preceding exemplary embodiments, wherein the constituent layer's velocity when the constituent layer contacts an interior wall of the collection container is substantially less than the velocity of the constituent layer as it exits the tube.
45. The device according to any one of the preceding exemplary embodiments, wherein the tube discharges into the collection container at a direction that is substantially parallel to the direction of the perimeter of the interior wall of the collection container.
46. The device according to any one of the preceding exemplary embodiments, wherein the tube discharges into the collection container at a direction that is substantially parallel to the direction of the perimeter of the interior wall of the collection container along the same radius from the axis.
47. The device according to any one of the preceding exemplary embodiments, wherein the tip of the tube traces a path as it rotates around the axis that it substantially parallel to an interior wall of the collection container.
48. The device according to any one of the preceding exemplary embodiments, wherein the direction of discharge out of the tube is substantially tangential to a circle centered about the axis in the plane of the circle.
49. The device according to any one of the preceding exemplary embodiments, wherein the direction of discharge out of the tube is substantially parallel to a tangent line of an interior wall of the collection container along the same radial direction from the axis.
50. The device according to any one of the preceding exemplary embodiments, wherein the distance from the end of the tube to the nearest wall of the collection container along the direction of discharge out of the tube is at least 3, 4, 5, 10, 15, 20, or 25 times the internal diameter of the end of the tube at a given position of the tube about the axis.
51. The device according to any one of the preceding exemplary embodiments, wherein the distance from the end of the tube to the nearest wall of the collection container along the direction of discharge out of the tube is at most 200, 150, 100, 75, 50, 25, 20, 15, or 10 times the internal diameter of the end of the tube at a given position of the tube about the axis.
52. The device according to any one of the preceding exemplary embodiments, wherein the distance from the end of the tube to the nearest wall of the collection container along the direction of discharge out of the tube is at least 3, 4, 5, 10, 15, 20, or 25 times the internal diameter of the end of the tube at any given position of the tube about the axis.
53. The device according to any one of the preceding exemplary embodiments, wherein the distance from the end of the tube to the nearest wall of the collection container along the direction of discharge out of the tube is at most 200, 150, 100, 75, 50, 25, 20, 15, or 10 times the internal diameter of the end of the tube at any given position of the tube about the axis.
54. The device according to any one of the preceding exemplary embodiments, wherein the velocity of the constituent layer at the time it contacts a wall of the collection container is 90% or less, 80% or less, 70% or less, 60% or less, 50% or less, 40% or less, or 30% or less of the velocity of the constituent layer at the point it is discharged from the tube.
55. The device according to any one of the preceding exemplary embodiments, wherein the velocity of the constituent layer at the time it contacts a wall of the collection container is from 20%, 30%, 40%, 50%, 60%, 70%, or 80% to 90% of the velocity of the constituent layer at the point it is discharged from the tube.
56. The device according to any one of the preceding exemplary embodiments, wherein the distance from the end of the tube to the nearest wall of the collection container along the direction of discharge out of the tube is at least 15 mm, 20 mm, 25 mm, 30 mm, 35 mm, 40 mm, 45 mm, 50 mm, 55 mm, 60 mm, 65 mm, 70 mm, or 75 mm.
57. The device according to any one of the preceding exemplary embodiments, wherein the internal cross-sectional area of the tube is greater at its exit than at its entrance.
58. The device according to any one of the preceding exemplary embodiments, wherein the internal cross-sectional area of the tube increases over its length.
59. The device according to any one of the preceding exemplary embodiments, wherein the internal cross-sectional area of the tube increases at a uniform rate over its length.
60. The device according to any one of the preceding exemplary embodiments, wherein the internal cross-sectional area of the tube is uniform over its length.
61. The device according to any one of the preceding exemplary embodiments, wherein the internal cross-sectional area of the first outlet is less than the internal cross-sectional area at the exit of the tube.
62. The device according to any one of the preceding exemplary embodiments, wherein the internal cross-sectional area of the first port is less than the internal cross-sectional area at the exit of the tube.
63. The device according to any one of the preceding exemplary embodiments, wherein the tube comprises a first portion extending in a direction that is at least radially away from the axis.
64. The device according to any one of the preceding exemplary embodiments, wherein the tube comprises a second portion that extends in a direction that is at least axially along the axis.
65. The device according to any one of the preceding exemplary embodiments, wherein the first portion and the second portion of the tube are separated by a bend.
66. The device according to any one of the preceding exemplary embodiments, wherein the first portion and the second portion of the tube are separated by a bend of approximately 90 degrees.
67. The device according to any one of the preceding exemplary embodiments, wherein the first portion extends from the entrance of the tube.
68. The device according to any one of the preceding exemplary embodiments, wherein the first portion extends to the exit of the tube.
69. The device according to any one of the preceding exemplary embodiments, wherein the first portion extends in a direction that is radially away from the axis.
70. The device according to any one of the preceding exemplary embodiments, wherein the first portion extends in a direction that is both radially away from the axis and axially along the axis.
71. The device according to any one of the preceding exemplary embodiments, wherein the second portion extends in a direction that is both radially away from the axis and axially along the axis.
72. The device according to any one of the preceding exemplary embodiments, wherein the second portion extends in a circular direction.
73. The device according to any one of the preceding exemplary embodiments, wherein the second portion extends in a direction circularly around the axis.
74. The device according to any one of the preceding exemplary embodiments, wherein the second portion extends in a circular direction and axially along the axis.
75. The device according to any one of the preceding exemplary embodiments, wherein the second portion extends in a circular direction, axially along the axis, and radially away from the axis.
76. The device according to any one of the preceding exemplary embodiments, wherein the tube extends in a direction axially along the axis along the entire length of the tube.
77. The device according to any one of the preceding exemplary embodiments, wherein the first portion extends in a direction radially away from the axis and axially along the axis and the second portion extends in a direction circularly around the axis and axially along the axis.
78. The device according to any one of the preceding exemplary embodiments, wherein the first portion extends in a direction radially away from the axis and axially along the axis and the second portion extends in a circular direction and axially along the axis.
79. The device according to any one of the preceding exemplary embodiments, wherein the first portion and the second portion overlap.
80. The device according to any one of the preceding exemplary embodiments, wherein the first portion and the second portion do not overlap.
81. The device according to any one of the preceding exemplary embodiments, further comprising a first valve operable to control fluid communication from the first outlet to the tube.
82. The device according to any one of the preceding exemplary embodiments, further comprising a first valve operable to control fluid communication from the first outlet to the tube, wherein the at least one of the constituent layers is dischargeable from the rotatable chamber via the tube and into the collection container by operating the valve.
83. The device according to any one of the preceding exemplary embodiments, wherein the first valve is openable by manual or electronic action.
84. The device according to any one of the preceding exemplary embodiments, wherein the first valve is openable in response to the centrifugal force exerted on it by rotation of the chamber.
85. The device according to any one of the preceding exemplary embodiments, wherein the first valve is opened by moving the first port or first outlet and the entrance of the tube into alignment.
86. The device according to any one of the preceding exemplary embodiments, wherein the first valve is manually operable by the user.
87. The device according to any one of the preceding exemplary embodiments, wherein the first valve operates automatically as a result of the operation of the device.
88. The device according to any one of the preceding exemplary embodiments, wherein the first valve comprises a dynamic seal.
89. The device according to any one of the preceding exemplary embodiments, wherein the dynamic seal is normally in the closed position, thereby restricting flow to the tube, and opens in response to the force exerted on the dynamic seal.
90. The device according to any one of the preceding exemplary embodiments, wherein the dynamic seal comprises an o-ring positioned in the first outlet or first port.
91. The device according to any one of the preceding exemplary embodiments, wherein the collection container is contained within the device.
92. The device according to any one of the preceding exemplary embodiments, wherein the collection container is contained within an outer enclosure.
93. The device according to any one of the preceding exemplary embodiments, wherein the collection container is separately removable from the device.
94. The device according to any one of the preceding exemplary embodiments, wherein the collection container is rotatable along with the rotatable chamber.
95. The device according to any one of the preceding exemplary embodiments, wherein the collection container is not configured to rotate along with the rotatable chamber.
96. The device according to any one of the preceding exemplary embodiments, wherein the tube rotates around the axis relative to the collection container.
97. The device according to any one of the preceding exemplary embodiments, wherein the collection container is cylindrical.
98. The device according to any one of the preceding exemplary embodiments, wherein the collection container is an elliptic cylinder.
99. The device according to any one of the preceding exemplary embodiments, wherein the collection container comprises a first interior wall and a second interior wall, wherein the radial distance from the axis of the first interior wall is less than the radial distance from the axis of the second interior wall.
100. The device according to any one of the preceding exemplary embodiments, wherein the collection container has an interior volume and the tube is configured to discharge into the interior volume of the collection container.
101. The device according to any one of the preceding exemplary embodiments, wherein the tube protrudes from a wall of the collection container.
102. The device according to any one of the preceding exemplary embodiments, wherein the rotatable chamber comprises a first outlet at a first radial distance from the axis, a retaining or morselizing screen at a second radial distance from the axis, a wiper at a third radial distance from the axis, and a fiber collector having an outer edge at a fourth radial distance from the axis, wherein the fourth radial distance is smaller than the third radial distance, the third radial distance is smaller than the second radial distance, and the second radial distance is smaller than the first radial distance.
103. The device according to any one of the preceding exemplary embodiments, wherein the rotatable chamber further comprises a second outlet having an entrance at a different radial distance from the axis than the entrance to the first outlet.
104. The device according to any one of the preceding exemplary embodiments, wherein the device further comprises a second valve associated with the second outlet.
105. The device according to any one of the preceding exemplary embodiments, wherein the device comprises one or more vents to allow for fluid displacement in the rotatable chamber.
106. The device according to any one of the preceding exemplary embodiments, wherein the inner chamber comprises a trap.
107. The device according to any one of the preceding exemplary embodiments, wherein the rotatable chamber comprises an oil trap for trapping oil near the base of the rotatable chamber.
108. The device according to any one of the preceding exemplary embodiments, wherein a filter is positioned at a distance above the base of the rotatable chamber, thereby forming a trap.
109. The device according to any one of the preceding exemplary embodiments, wherein the filter is shaped like a disc.
110. The device according to any one of the preceding exemplary embodiments, wherein the filter has an average opening size is of at least 0.025 mm or 0.05 mm.
111. The device according to any one of the preceding exemplary embodiments, wherein the filter to has an average opening size of at most 0.25 mm, 0.2 mm, 0.15 mm, or 0.13 mm.
112. The device according to any one of the preceding exemplary embodiments, further comprising a fiber collector.
113. The device according to any one of the preceding exemplary embodiments, wherein the fiber collector is configured to collect fibers from a sample.
114. The device according to any one of the preceding exemplary embodiments, wherein the fiber collector is rotatable.
115. The device according to any one of the preceding exemplary embodiments, wherein the fiber collector is rotatable about its central axis.
116. The device according to any one of the preceding exemplary embodiments, wherein the fiber collector is rotatable about the axis of the rotatable chamber.
117. The device according to any one of the preceding exemplary embodiments, wherein the fiber collector is connected to the same rotation source as the rotatable chamber.
118. The device according to any one of the preceding exemplary embodiments, wherein the fiber collector is connected to the same rotation source as the rotatable chamber and is configured to rotate slower than the rotation source of the rotatable chamber.
119. The device according to any one of the preceding exemplary embodiments, wherein fiber collector is connected to a different rotation source than the rotation source connected to the rotatable chamber.
120. The device according to any one of the preceding exemplary embodiments, wherein fiber collector is connected to its own rotation source.
121. The device according to any one of the preceding exemplary embodiments, wherein the rotation source coupled to the fiber collector is present at the top of the rotatable chamber and may be decoupled from the fiber collector and reused.
122. The device according to any one of the preceding exemplary embodiments, wherein the fiber collector has the same revolution frequency as the rotatable chamber.
123. The device according to any one of the preceding exemplary embodiments, wherein the fiber collector is configured to rotate and at a slower revolution frequency than the rotatable chamber.
124. The device according to any one of the preceding exemplary embodiments, wherein the fiber collector is configured to rotate at a faster revolution frequency than the rotatable chamber.
125. The device according to any one of the preceding exemplary embodiments, wherein the fiber collector is present coaxially with or proximate to the axis of the rotatable chamber.
126. The device according to any one of the preceding exemplary embodiments, wherein the fiber collector is present at a fifth radial distance from the axis of rotation of the rotatable chamber.
127. The device according to any one of the preceding exemplary embodiments, wherein the fiber collector comprises a plurality of arms into which one or more fibers of tissue may be captured.
128. The device according to any one of the preceding exemplary embodiments, wherein the fiber collector is configured to rotate about the axis of rotation of the rotatable chamber.
129. The device according to any one of the preceding exemplary embodiments, wherein the fiber collector comprises a plurality of arms extending away from the axis.
130. The device according to any one of the preceding exemplary embodiments, wherein the fiber collector comprises a plurality of arms extending radially from the axis.
131. The device according to any one of the preceding exemplary embodiments, wherein the fiber collector comprises a plurality of entangled arms.
132. The device according to any one of the preceding exemplary embodiments, wherein the fiber collector comprises a plurality of wires.
133. The device according to any one of the preceding exemplary embodiments, wherein the fiber collector comprises a plurality of bristles.
134. The device according to any one of the preceding exemplary embodiments, wherein the arms of the fiber collector extend straight away from the axis.
135. The device according to any one of the preceding exemplary embodiments, wherein the fiber collector is a brush.
136. The device according to any one of the preceding exemplary embodiments, wherein the fiber collector is a comb.
137. The device according to any one of the preceding exemplary embodiments, wherein the fiber collector comprises a helix brush.
138. The device according to any one of the preceding exemplary embodiments, wherein the fiber collector comprises a cylindrical brush.
139. The device according to any one of the preceding exemplary embodiments, wherein the height of the fiber collector should be at least as high as the amount of sample delivered to the rotatable chamber.
140. The device according to any one of the preceding exemplary embodiments, wherein the fiber collector has an outer diameter of 2 inches (5.08 cm) or less, 1.5 inches (3.81 cm) or less, 1 inch (2.54 cm) or less, 0.75 inches (1.91 cm) or less, or 0.5 inches (1.27 cm) or less.
141. The device according to any one of the preceding exemplary embodiments, wherein the fiber collector is removable from the rotatable chamber.
142. The device according to any one of the preceding exemplary embodiments, wherein the fiber collector may be uncoupled from a cap that closes off the rotatable chamber and removed from the rotatable chamber.
143. The device according to any one of the preceding exemplary embodiments, wherein the rotatable chamber further comprises a wiper.
144. The device according to any one of the preceding exemplary embodiments, wherein the wiper disrupts fibers hanging off of the fiber collector to cause the desirable fat tissue to be released from the fibers.
145. The device according to any one of the preceding exemplary embodiments, wherein the wiper is a cylindrical rod.
146. The device according to any one of the preceding exemplary embodiments, wherein the wiper is present proximate the fiber collector in a position that the wiper will not contact the fiber collector during operation of the device.
147. The device according to any one of the preceding exemplary embodiments, wherein the fiber collector is coaxial to the axis of rotation of the rotatable chamber and the wiper is present at a radius from the axis that is greater than the outermost radius of the fiber collector.
148. The device according to any one of the preceding exemplary embodiments, wherein the fiber collector is rotatable while the wiper remains stationary.
149. The device according to any one of the preceding exemplary embodiments, wherein the wiper is rotatable while the fiber collector remains stationary.
150. The device according to any one of the preceding exemplary embodiments, wherein the wiper is rotatable about the fiber collector at a different revolution frequency than the fiber collector.
151. The device according to any one of the preceding exemplary embodiments, further comprising a retaining screen.
152. The device according to any one of the preceding exemplary embodiments, further comprising a retaining screen configured to restrict the passage of a constituent of biologic tissue therethrough, wherein the retaining screen meets the second end or sidewall at a second radial distance from the axis, and wherein the second radial distance is less than the first radial distance.
153. The device according to any one of the preceding exemplary embodiments, wherein the retaining screen projects from the second end.
154. The device according to any one of the preceding exemplary embodiments, wherein the retaining screen projects away from the base.
155. The device according to any one of the preceding exemplary embodiments, wherein the retaining screen is configured to substantially retain a certain type of tissue on the interior of the retaining screen while allowing liquids to pass through.
156. The device according to any one of the preceding exemplary embodiments, wherein the retaining screen is configured to retain fat tissue on its inner surface.
157. The device according to any one of the preceding exemplary embodiments, wherein the retaining screen is cylindrical.
158. The device according to any one of the preceding exemplary embodiments, wherein a surface of the retaining screen is substantially parallel to the inner surface of the sidewall.
159. The device according to any one of the preceding exemplary embodiments, wherein the retaining screen is frustoconical.
160. The device according to any one of the preceding exemplary embodiments, wherein the retaining screen rotates along with the rotatable chamber.
161. The device according to any one of the preceding exemplary embodiments, wherein the retaining screen is stationary relative to the rotatable chamber.
162. The device according to any one of the preceding exemplary embodiments, wherein the retaining screen rotates at a revolution frequency that is less that the revolution frequency of the rotatable chamber.
163. The device according to any one of the preceding exemplary embodiments, wherein the retaining screen is in contact with the second end.
164. The device according to any one of the preceding exemplary embodiments, wherein the retaining screen is in contact with the sidewall.
165. The device according to any one of the preceding exemplary embodiments, wherein the retaining screen is in contact with the first end.
166. The device according to any one of the preceding exemplary embodiments, wherein the retaining screen extends concentrically around the axis of the rotatable chamber.
167. The device according to any one of the preceding exemplary embodiments, wherein the retaining screen is mesh-like.
168. The device according to any one of the preceding exemplary embodiments, wherein the retaining screen is made of metal or polymer wire material, or a perforated sheet having openings of sufficient size to allow for the passage of fluid while inhibiting desirable material to pass through.
169. The device according to any one of the preceding exemplary embodiments, wherein the first outlet is located further from the axis than the retaining screen, such that material must pass through the retaining screen to reach the first outlet.
170. The device according to any one of the preceding exemplary embodiments, further comprising a morselizing screen.
171. The device according to any one of the preceding exemplary embodiments, further comprising a morselizing screen configured to morselize a biologic tissue into smaller fragments, wherein the morselizing screen meets the second end at a second radial distance from the axis, and wherein the second radial distance is less than the first radial distance.
172. The device according to any one of the preceding exemplary embodiments, wherein the morselizing screen is used to morselize tissue into smaller fragments.
173. The device according to any one of the preceding exemplary embodiments, wherein the morselizing screen is configured to break a plurality of adipocytes present in adipose tissue.
174. The device according to any one of the preceding exemplary embodiments, wherein the morselizing screen is present farther from the axis of rotation of the rotatable chamber than the fiber collector but closer to the axis than the retaining screen.
175. The device according to any one of the preceding exemplary embodiments, wherein the morselizing screen is located at a sixth radial distance, that is less than the second radial distance and greater than the third radial distance from the axis.
176. The device according to any one of the preceding exemplary embodiments, wherein the morselizing screen is cylindrical.
177. The device according to any one of the preceding exemplary embodiments, wherein a surface of the morselizing screen is substantially parallel to the inner surface of the sidewall.
178. The device according to any one of the preceding exemplary embodiments, wherein the morselizing screen is frustoconical.
179. The device according to any one of the preceding exemplary embodiments, wherein the morselizing screen rotates along with the rotatable chamber.
180. The device according to any one of the preceding exemplary embodiments, wherein the morselizing screen is stationary relative to the rotatable chamber.
181. The device according to any one of the preceding exemplary embodiments, wherein the morselizing screen rotates at a revolution frequency that is less that the revolution frequency of the rotatable chamber.
182. The device according to any one of the preceding exemplary embodiments, wherein the morselizing screen projects away from the base.
183. The device according to any one of the preceding exemplary embodiments, wherein the morselizing screen extends concentrically around the axis of rotation of the rotatable chamber.
184. The device according to any one of the preceding exemplary embodiments, wherein the morselizing screen is mesh-like.
185. The device according to any one of the preceding exemplary embodiments, wherein the retaining screen is made of metal or polymer wire material, or a perforated sheet having openings of sufficient size to allow for the morselization of a tissue.
186. The device according to any one of the preceding exemplary embodiments, wherein the morselizing screen is in contact with the second end.
187. The device according to any one of the preceding exemplary embodiments, wherein the morselizing screen is in contact with the sidewall.
188. The device according to any one of the preceding exemplary embodiments, wherein the morselizing screen is in contact with the first end.
189. The device according to any one of the preceding exemplary embodiments, further comprises a roller arranged to urge tissue through the morselizing screen.
190. The device according to any one of the preceding exemplary embodiments, wherein the roller comprises a roller axle and a cylinder rotatable about the roller axle.
191. The device according to any one of the preceding exemplary embodiments, wherein the roller axle is formed of stiff wire that extends through the center bore of the cylinder and the wire is mounted so that it may be secured in a stationary position within the inner chamber.
192. The device according to any one of the preceding exemplary embodiments, wherein the roller is present proximate or in contact with the morselizing screen.
193. The device according to any one of the preceding exemplary embodiments, wherein the morselizing screen is rotatable about the axis of rotation of the rotatable chamber while the roller axle remains stationary.
194. The device according to any one of the preceding exemplary embodiments, wherein the roller is present near the inner surface of the morselizing screen but does not contact the morselizing screen.
195. The device according to any one of the preceding exemplary embodiments, wherein the roller rolls against the inner surface of the morselizing screen.
196. The device according to any one of the preceding exemplary embodiments, wherein the drive unit couples to the rotatable chamber.
197. The device according to any one of the preceding exemplary embodiments, wherein the drive unit comprises an electric motor configured to rotate the rotatable chamber
198. The device according to any one of the preceding exemplary embodiments, wherein the drive unit comprises a hand crank to rotate the rotatable chamber.
199. The device according to any one of the preceding exemplary embodiments, wherein the drive unit comprises a hand crank and a spring and is configured so that the drive unit can be would up and the spring released to rotate the rotatable chamber.
200. The device according to any one of the preceding exemplary embodiments, wherein the drive unit is separable from the rotatable chamber such that the drive unit may be reused without substantial cleaning or sterilization.
201. The device according to any one of the preceding exemplary embodiments, wherein the rotatable chamber is single-use or may be cleaned and sterilized, such that it may be reused.
202. The device according to any one of the preceding exemplary embodiments, wherein the drive unit is configured to rotate the rotatable chamber at multiple speeds.
203. The device according to any one of the preceding exemplary embodiments, wherein the drive unit is configured to rotate the rotatable chamber such that the g-force acting on a sample within the rotatable chamber is 1000 g or less, 8000 g or less, or 5000 g or less.
204. The device according to any one of the preceding exemplary embodiments, wherein the drive unit is configured to oscillate the rotatable chamber.
205. The device according to any one of the preceding exemplary embodiments, wherein the drive unit is configured to oscillate in a sinusoidal wave pattern.
206. The device according to any one of the preceding exemplary embodiments, wherein the drive unit is configured to oscillate in a square wave pattern.
207. The device according to any one of the previous exemplary embodiments, wherein the device is configured to allow for addition of a cleaning solution while the rotatable chamber is rotating.
208. A process for extracting a constituent from a sample of biologic tissue comprising the steps of:
   a. introducing a sample into a rotatable chamber,
   b. rotating the rotatable chamber about an axis, thereby producing a centrifugal field sufficient to stratify the sample into at least two constituents based on the density of the constituents,
   c. operating a valve to discharge a first fluid constituent of the sample from a first outlet of the rotatable chamber via a tube connected to the first outlet and into a collection container,
   wherein the exit of the tube and the collection container are configured such that the direction of discharge out of the tube is substantially tangential to a circle centered about the axis in the plane of the circle.
209. A process for extracting a constituent from a sample of biologic tissue comprising the steps of:
   a. introducing a sample into a rotatable chamber,
   b. rotating the rotatable chamber about an axis, thereby producing a centrifugal field sufficient to stratify the sample into at least two constituents based on the density of the constituents,
   c. operating a valve to discharge a first fluid constituent of the sample from a first outlet of the rotatable chamber via a tube connected to the first outlet and into a collection container,
   wherein an interior wall of the collection container and the trajectory of the exit of the tube are configured to reduce the velocity of the constituent layer at the time the constituent layer contacts the interior wall of the collection container relative to the velocity of the constituent layer at the time that the constituent layer exits the tube.
210. A process for processing a sample of biologic tissue comprising the steps of:
   a. providing a device according to any one of the preceding exemplary embodiments,
   b. introducing a sample into the rotatable chamber,
   c. rotating the rotatable chamber about an axis, thereby stratifying the sample into at least two constituents based on the density of the constituents,
   d. removing a first fluid constituent from the sample via the first outlet in the rotatable chamber; and
   e. discharging the first fluid constituent into a collection container.
211. The process according to the previous exemplary embodiment, further comprising the step of recovering the desired portion of the sample from the collection container.
212. The process according to any one of the previous exemplary embodiments, further comprising the step of adding a cleaning solution after removing a first fluid constituent from the sample and prior to recovering the desired constituent of the sample.
213. The process according to any one of the previous exemplary embodiments, further comprising the step of adding a cleaning solution to the rotatable chamber while the rotatable chamber is rotating.
214. The process according to any one of the previous exemplary embodiments, further comprising the step of oscillating the rotatable chamber after adding the cleaning solution.
215. The process according to any one of the previous exemplary embodiments, further comprising the step of rotating the rotatable chamber, thereby stratifying the sample into at least two constituents based on the density of the constituents, and removing a second fluid constituent from the sample via the second outlet in the rotatable chamber, wherein the second fluid constituent comprises the cleaning solution.
216. The process according to any one of the previous exemplary embodiments, wherein the process further comprises the step of capturing the fibrous content of the sample by rotating a fiber collector.
217. The process according to any one of the previous exemplary embodiments, further comprising the step of retaining the biologic tissue on a retaining screen.
218. The process according to any one of the previous exemplary embodiments, further comprising the step of morselizing the biologic tissue.
219. The process according to any one of the previous exemplary embodiments, further comprising the step of urging the biologic tissue through a morselizing screen, thereby morselizing the biologic tissue.
220. The process according to any one of the preceding exemplary embodiments, further comprising the step of reducing the velocity of the first fluid constituent such that the constituent layer's velocity when the constituent layer contacts a wall of the collection container is less than the velocity of the constituent layer as it exits the tube.
221. The process according to any one of the preceding exemplary embodiments, further comprising the step of reducing the velocity of the first fluid constituent such that the constituent layer's velocity when the constituent layer contacts a wall of the collection container is less than the velocity of the constituent layer as it enters the collection container.
222. The process according to any one of the previous exemplary embodiments, wherein the process is performed utilizing the device of any one of the previous exemplary embodiments.

1. A device for processing biologic tissue, the device comprising:
a. a rotatable chamber arranged to rotate around an axis, the rotatable chamber comprising a first end, a second end, a sidewall extending between the first end and the second end and comprising an inner surface that decreases in diameter over at least a portion of the distance between the first end and the second end, and a first outlet, the first outlet beginning in the interior of the rotatable chamber at a first radial distance from the axis;
b. a tube connected or connectable to the first outlet, the tube extending in a direction that is at least radially away from the axis over at least part of the tube's length;
c. a collection container in fluid communication with the tube; and
d. a drive unit configured to rotate the rotatable chamber about the axis;
wherein the device is operable such that a centrifugal field is produced via rotation of the rotatable chamber about the axis, thereby causing a sample of biologic tissue present in the rotatable chamber to stratify into at least two constituent layers as a function of the differing specific gravities of the constituents, at least one of the constituent layers being dischargeable from the rotatable chamber via the tube and into the collection container,
wherein an interior wall of the collection container and the trajectory of the exit of the tube are configured such that the direction of discharge out of the tube is substantially parallel to a tangent line of a wall of the collection container along the same radius from the axis.

2. The device according to claim 1, further comprising a first valve operable to control fluid communication from the first outlet to the tube, wherein the at least one of the constituent layers is dischargeable from the rotatable chamber via the tube and into the collection container by operating the valve.

3. The device according to claim 1, wherein the tube is connected or connectable to the first outlet and at a distance greater than the first radial distance.

4. The device according to claim 1, wherein the device is configured such that the tube rotates along with the rotatable chamber.

5. The device according to claim 1, wherein the constituent layer's velocity when the constituent layer contacts a wall of the collection container is substantially less than the velocity of the constituent layer as it exits the tube.

6. The device according to claim 1, wherein the tube discharges into the collection container at a direction that is substantially parallel to the direction of the perimeter of the wall of the collection container along the same radius from the axis.

7. The device according to claim 1, wherein the tip of the tube rotates along with the rotatable chamber and the tube traces a path as it rotates around the axis that it substantially parallel to an interior wall of the collection container.

8. The device according to claim 1, wherein the direction of discharge out of the tube is substantially tangential to a circle centered about the axis in the plane of the circle.

9. The device according to claim 1, wherein the distance from the end of the tube to the nearest wall of the collection container along the direction of discharge out of the tube is from 5 to 200 times the internal diameter of the end of the tube at any given position of the tube about the axis.

10. The device according to claim 1, wherein the velocity of the constituent layer at the time it contacts a wall of the collection container is 90% or less of the velocity of the constituent at the point it is discharged from the tube.

11. The device according to claim 1, wherein the distance from the end of the tube to the nearest wall of the collection container along the direction of discharge out of the tube is at least 15 mm.

12. The device according to claim 1, wherein the tube comprises a first portion extending in a direction that is at least radially away from the axis and a second portion that extends in a direction that is at least axially along the axis.

13. The device according to claim 1, wherein the tube comprises a portion that extends in a direction that is both radially away from the axis and axially along the axis.

14. The device according to claim 1, wherein the tube comprises a portion that extends in a direction substantially circularly around the axis.

15. A process for processing a sample of biologic tissue comprising the steps of:
a. providing a device according to claim 1,
b. introducing a sample into the rotatable chamber,
c. rotating the rotatable chamber, thereby stratifying the sample into at least two constituents based on the density of the constituents,
d. removing a first fluid constituent from the sample via the first outlet in the rotatable chamber; and
e. discharging the first fluid constituent into a collection container.

16. A process for extracting a constituent from a sample of biologic tissue comprising the steps of:
a. introducing a sample into a rotatable chamber,
b. rotating the rotatable chamber about an axis, thereby producing a centrifugal field sufficient to stratify the sample into at least two constituents based on the density of the constituents,
c. operating a valve to discharge a first fluid constituent of the sample from a first outlet of the rotatable chamber via a tube connected to the first outlet and into a collection container,
wherein the exit of the tube and the collection container are configured such that the direction of discharge out of the tube is substantially tangential to a circle centered about the axis in the plane of the circle.

17. The process according to claim 16, further comprising the step of morselizing the biologic tissue.

18. The process according to claim 16, further comprising the step of removing a second fluid constituent from the sample via a second outlet in the rotatable chamber.

19. The process according to claim 18, wherein the second fluid constituent comprises a cleaning solution.

20. The process according to claim 17, further comprising the step of retaining the biologic tissue on a retaining screen.
